(19) United States
(12) Patent Application Publication
(10) Pub. No.: US 2015/0037810 A1
(43) Pub. Date: Feb. 5, 2015

(54) DISEASE PATHWAY-BASED METHOD TO GENERATE BIOMARKER PANELS TAILORED TO SPECIFIC THERAPEUTICS FOR INDIVIDUALIZED TREATMENTS

(71) Applicant: Peter Blume-Jensen, Westfield, NJ (US)

(72) Inventor: Peter Blume-Jensen, Westfield, NJ (US)

(21) Appl. No.: 13/954,080

(22) Filed: Jul. 30, 2013

(51) Int. Cl. G01N 33/50

(52) U.S. Cl. CPC G01N 33/5041
USPC 435/7.1

(57) ABSTRACT

The increased efficacy and reduced unwanted side effects of drugs can be insured by treating only responsive patients. In an embodiment of the invention, signaling pathways that a particular drug interferes with, are derive together with predictive biomarkers and dynamic biomarker that can read the activity of these pathways before and after drug treatment in order to select a responder patient population. In an alternative embodiment of the invention, certain core pathways that the drug does not interfere with and that are known to be causally involved in a particular disease(s) can be identified, and derive the biomarkers for those to be able to exclude these patients that suffer from a disease in which those drug non effected pathways are involved from being treated with the specific drug in question.

### PRIORITY CLAIM

This application claims priority under 35 U.S.C. 119(e) to U.S. Provisional Patent Application No. 61/013,249, filed Dec. 12, 2007; and to U.S. patent application Ser. No. 12/331,356 filed Dec. 9, 2008, which are incorporated herein by reference.

### FIELD OF THE INVENTION

The present invention relates to methods of treating diseases based on identifying and establishing disease pathway-oriented biomarkers and biomarker tools that semi-quantitatively measure the effect and intersection point of specific therapeutics on disease pathways and that are also predictive for efficacy in treating specific patient populations with a particular exogenous substance, including, but not limited to biologics, biologics-derived, and synthetic therapeutics.

### BACKGROUND OF THE INVENTION

During the last decade an increasing number of so-called ‘Targeted Therapeutics’ have been developed. These are treatments directed to correct or abrogate the underlying molecular defects driving specific diseases while causing only minimal unwanted effects. However, most diseases are molecularly heterogeneous, and so only a fraction of patients with a certain disease share an underlying molecular disease mechanism. As a consequence, pharmaceutical and biotech companies are now facing an enormous challenge: how to identify the specific sub-group of patients with a certain disease that are likely to respond to their specific targeted therapeutic.

In the field of oncology, more than 220 targeted therapeutics are currently in clinical development, and it is predicted that less than 9% of these will make it to the market. This is primarily due to the inability to predict efficacy and identify responders. Hence, the biggest attrition occurs in phase IIB, which is the stage of clinical trials where efficacy is assessed. It takes on average 7 years to bring a new project through successful phase IIB, and a failed phase IIB oncology drug costs on average $M 150-280. Also in other therapeutic areas the attrition rate for therapeutics is highest in Phase IIB clinical trials.

### BRIEF DESCRIPTION OF THE FIGURES

The details of one or more embodiments are set forth in the description below. Other features, objects and advantages will be apparent from the description, the drawings, and the claims.

FIG. 1 is a schematic showing the proposed approach according to an embodiment of the invention;

FIG. 2 is a schematic showing a classical classification of cancers based on the anatomical tissue of origin of specific cancer types;

FIG. 3 is a schematic showing a new classification of cancers based on the various pathway alterations that are involved in specific cancer types based on an embodiment of the invention;

FIG. 4 shows a representative flow scheme of the processes used to identify and generate pathway-based biomarkers for a specific cancer therapeutic;

FIG. 5 illustrates an example of a therapeutic that inhibits two different signaling pathways and consequent alterations in signaling pathway activity distally of the drug interception level as measured using phospho-specific antibodies

FIG. 6 shows an example of two dynamic phosphoantibody biomarkers measuring the drug target exposure, and two exclusion phosphoantibodies that indicate undesirable pathway activity;

FIG. 7 illustrates an example of a therapeutic that inhibits two different signaling pathways and consequent alterations in signaling pathway activity distally of the drug interception level as measured using phospho-proteomics or other pathway activation-state read-out;

FIG. 8 illustrates how the drug-induced pathway alterations provide basis for generating dynamic phosphoantibody biomarkers measuring the drug target exposure, as well as exclusion phosphoantibodies that indicate undesirable pathway activity;

FIG. 9 illustrates a real example of a malignant melanoma patient harboring different oncogenic mutations and who is being treated with a MEK inhibitor;

FIG. 10 illustrates the use of a dynamic phosphoantibody biomarker and exclusion phosphoantibodies for a malignant melanoma patient treated with a MEK inhibitor;

FIG. 11 illustrates a cell line which harbors a deregulated pathway causing a disease. Upon drug inhibition or genetic downregulation, an alternative pathway leading to the disease is activated;

FIG. 12 illustrates the cell line shown in FIG. 11 which harbors a deregulated pathway causing a disease. Despite drug inhibition or genetic downregulation, the same conserved disease-causing by-pass' mechanism can be activated as that shown in FIG. 11;

FIG. 13 illustrates the cell line shown in FIG. 12 which harbors the deregulated disease-causing pathway and how drug inhibition or genetic downregulation of targets block the main and alternative pathways leading to the disease; and

FIG. 14 shows a representative flow scheme of the processes used to identify and generate pathway-based biomarkers for a specific therapeutic.

### DETAILED DESCRIPTION OF THE INVENTION

Prior to setting forth the invention, it may be helpful to an understanding thereof to first set forth definitions of certain terms that are used hereinafter.

“Biomarker” means a molecule that indicates activity of a disease pathway. It is most typically, but not necessarily, translated from RNA. Increased abundance or specific post-translational modification of the Biomarker indicates that the disease signaling pathway activity has been up regulated. Decreased abundance or specific post-translational modification of the Biomarker indicates that the disease signaling pathway activity has been down regulated.

“Biomarker Responder Package” means a panel of biomarker predictive of response to a therapeutic and also dynamically regulated in response to the therapeutic. The package enables responder stratification in trials and target exposure monitoring of the therapeutic.

“Cancer Space” means most pathways which result in the cancer.

“Combinatorial Targeted Therapeutics” means a combination of targeted therapeutics that when used together have additive or synergistic treatment effect

“Disease” means a pathological condition of a mammal which leads to a debilitating condition of the mammal caused by a perturbation of a genetic pathway.

“Disease Space” means most pathways which result in the disease.

“Phosphoantibodies” are antibodies that are directed against and specifically recognize phosphorylation of a specific amino acid(s) in a specific amino acid sequence of a specific protein. Phosphorylation is one of several post-translational modifications and indicates the activity state in a disease pathway of a particular protein.

“Activation-state antibodies” are antibodies that are directed against and specifically recognize modifications of cellular molecules, most often proteins, that alter the activity-state of said molecule. These modifications are typically post-translational modifications and indicate the activity state in a disease pathway of a particular protein.

“Phosphoproteomics” is a mass spectrometry-based method to identify and semi-quantitatively or qualitatively measure the phosphorylation state of individual proteins within a pool of proteins.

“Phosphosignatures” are specific peptides residing within proteins in a cell, wherein one or more of the peptide residues is phosphorylated. The pattern of phosphorylated peptides in a cell constitutes a characteristic phosphosignature.

“Post-Translational Product” means the product of a RNA translation process that has been subsequently modified in a post-translational event.

“Translational Product” means the product of a RNA translation process.

“Responder Identification” means the ability to identify the patients that will be effectively treated with a particular drug or exogenous agent. This can be achieved by use of the ‘biomarker responder package’.

“Specific Disease” means a pathological condition caused by on one or several perturbed signaling pathways.

“Therapeutic” or “drug” means an exogenous agent intended to be administered to a diseased mammal. A drug that interferes with the signaling activity of a disease pathway involved in a specific disease, allows matching of the right drug with the right patients, based on a profiling of the drug's activity on said disease signaling pathways.

The inability to identify responder patients for targeted therapeutics that are active in smaller and smaller disease markets together with increasing costs for bringing drugs to market, has led to decreasing return of investment for Pharmaceutical companies. On top of that, patent expirations leading to generics competition and problems for Health Insurance companies to predict and reimburse patient treatment expenditures, has led to the realization of the urgent need for a method to stratify and segregate patients to ensure efficacy of therapeutics. In fact, there is a push from Health Insurance companies to have efficient methods to identify the patients that will respond to a therapeutic, and there are emerging examples of reimbursement being contingent on efficacy of a particular therapeutic. Finally, there is a huge emotional and societal impact with the present methodology where patients are being treated with in-efficient drugs.

Due to the challenges in making ‘block buster’ drugs, there is a need and desire for new and preferably improved therapeutics for defined patient populations. The key challenge for the successful market launch of these new targeted therapeutics is to be able to identify the responders, both during clinical trials and for drugs on the market.

In order to address the development of a therapeutic for a specific patient population, an approach is required that enables optimal selection of responder patients in much smaller phase I clinical trials. These trials can be designed to also assess response to the drug. This will enable much quicker go, no-go decisions, and ultimately result in more efficient therapeutic development thus reducing therapeutic development costs.

A related, but unique problem is that of identifying the optimal combination of targeted therapeutics that will provide maximal treatment efficacy for specific diseases. Most diseases are not possible to completely cure by interfering with just one molecular mechanism or target, but often several targets need to be modulated by several therapeutics to provide optimal treatment efficacy and prevent so-called drug resistance or ‘by-pass’ to kick in.

Previously, a genetics approach using genomics technologies has been used to correlate specific genetic alterations with specific disease phenotypes. While extremely powerful and able to identify most changes at the genetic level with specific diseases, these changes are associative and not necessarily causally involved in the disease phenotype. Hence, it is usually an enormous challenge to identify the best molecular drug targets through a genetic analysis. Consequently, relatively few successful predictive genetic biomarker approaches have been identified.

In an embodiment of the invention, the activity of specific intracellular signal transduction pathways can be linked with the observed disease phenotype. Since these pathways are the effectors of cellular behavior they are causally involved in the specific disease phenotype. In various embodiments of the invention, a pathway-based approach can be used for Identification of the appropriate Responder to a drug. In various embodiments of the invention, a pathway-based approach can be used for identification of optimal Combinatorial Targeted Therapeutics and the associated biomarker responder package. In an embodiment of the invention, a pathway-based approach can be used for rational selection of patients going into clinical trials. In an alternative embodiment of the invention, a pathway-based approach can be used for selecting patients that will be most effectively treated by one or more drugs. The pathway-based approach will allow stratification of patients and selection of only those patients responding to the right combinations of treatments.

### Definition of a Specific Disease

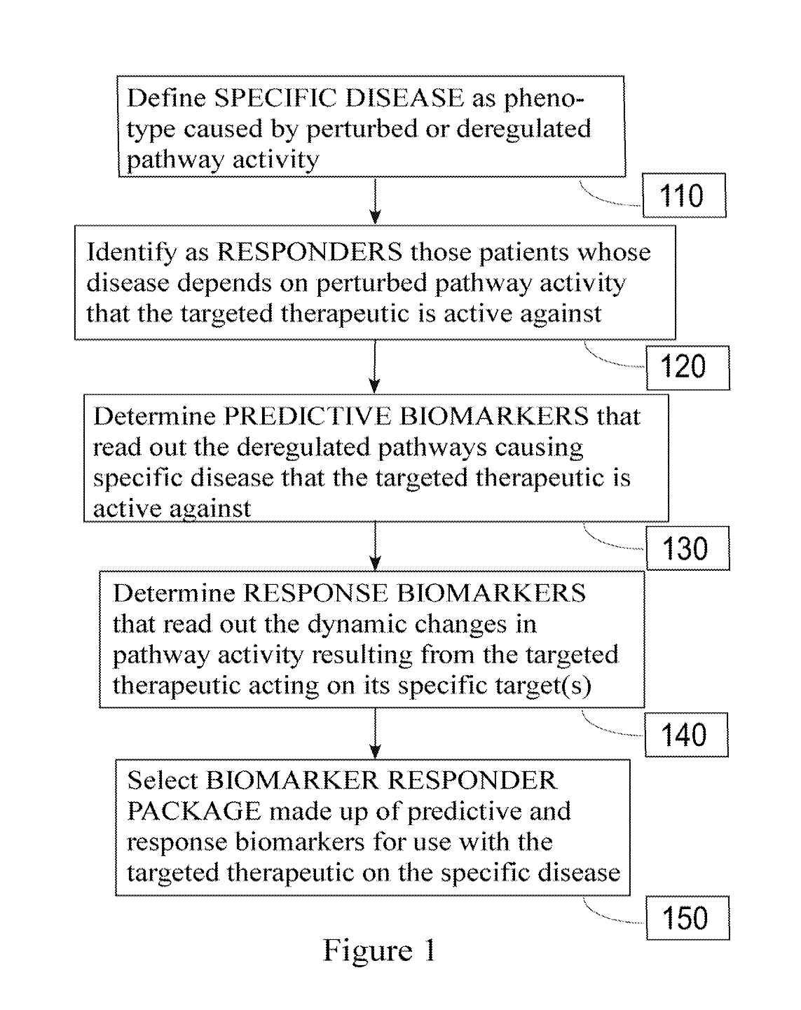

Rather than defining a specific disease based on the tissue of origin and its histo-pathological appearance, the specific disease 110 is defined based on the perturbed signaling pathways that cause the specific disease phenotype as shown in FIG. 1. Based on this pathway definition of disease, a specific disease can be classified into several sub-groups or fractions, each having a specific pattern of perturbed or deregulated pathway activity. For instance, specific types of cancer, like breast, colorectal or prostate, each can be stratified into several sub-groups that are characterized by a certain signaling pathway activity pattern. Likewise, specific types of inflammatory, auto-immune, neurological, and other diseases can be sub-grouped based on a shared deregulated or perturbed signaling pathway pattern within a specific sub-group of patients with a specific disease. Since the perturbed pathway activity pattern is causing the disease phenotype, it is possible to link the effect of a particular drug on the activity of specific signaling pathways with its ability to cause a desirable therapeutic effect. If the drug modulates perturbed pathway activity linked to a sub-fraction of patients 120 with a specific disease 110, that particular drug is expected to be effective in treating that sub-fraction of patients with the particular disease. Conversely, if the drug does not modulate the signaling activity of (an)other pathway(s) causally involved in the disease phenotype it is not expected to be effective for treating said patient sub-population.

### Predictive and Dynamic Biomarkers

Based on this concept, the signaling pathways that a particular drug interferes with, can be identified and the predictive biomarkers 130 and dynamic biomarkers 140 that can read the activity of these pathways before and after drug treatment can be derived (see FIG. 1). Likewise, certain core pathways that the drug does not interfere with and that are known to be causally involved in a particular disease(s) can be identified, and biomarkers derived for those pathways in order to be able to exclude these patients that suffer from a disease (in which those drug non effected pathways are involved) from being treated with the specific drug in question.

### Biomarker Responder Package

Thus for a specific drug, a Biomarker Responder Package 150 is made up of a collection or panel of predictive biomarkers 130 and dynamic biomarkers 140 that can be derived for use with a specific drug to act on the specific disease where the Biomarker Responder Package can read the activity of these pathways before and after drug treatment (see FIG. 1). In various embodiments of the invention, the predictive or dynamic biomarkers can be antibodies. In an embodiment of the invention, the predictive or dynamic biomarkers can be antibodies directed against phosphorylated or otherwise post-translationally modified proteins. For a specific drug, a simple constellation of phosphorylation state-specific or other activation state-specific antibodies can be derived and used to identify the key nodes of signaling activity that are compatible with beneficial therapeutic efficacy and also those that can preclude efficacy of the drug. In an embodiment of the invention, approximately 3 to approximately 20 activation-state antibodies can predict the response of a mammal to a therapeutic. In an embodiment of the invention, 4 to 8 activation-state antibodies can predict the response of a mammal to a therapeutic.

In alternative embodiments of the invention, a biomarker package can contain other tools in addition to, or instead of activation-state antibodies that are able to identify and directly or indirectly measure the level of activity of specific signaling pathways. Examples include, but are not limited to phosphoproteomics and other mass spec-based approaches, reporter assays based on chemiluminescence, fluorescence, radioactivity, and other reporter signal, degradation of signaling proteins by ubiquitination, and other proteasome-mediated processes, scaffold and chaperone protein cargo proteins.

### Method I. Identification, Generation, and Application of Predictive and Dynamic Biomarkers for a Specific Therapeutic.

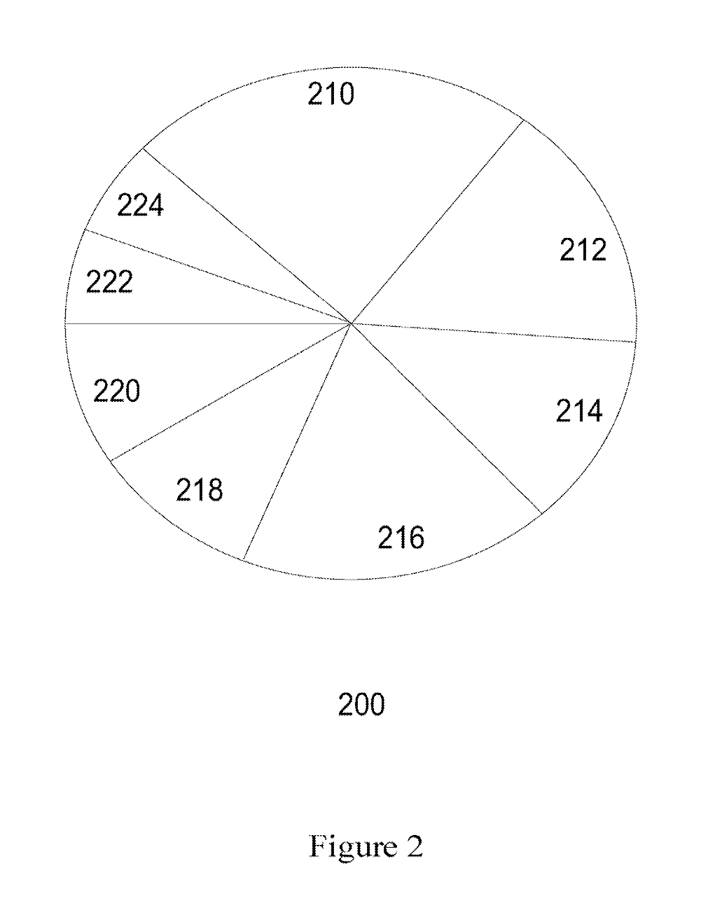

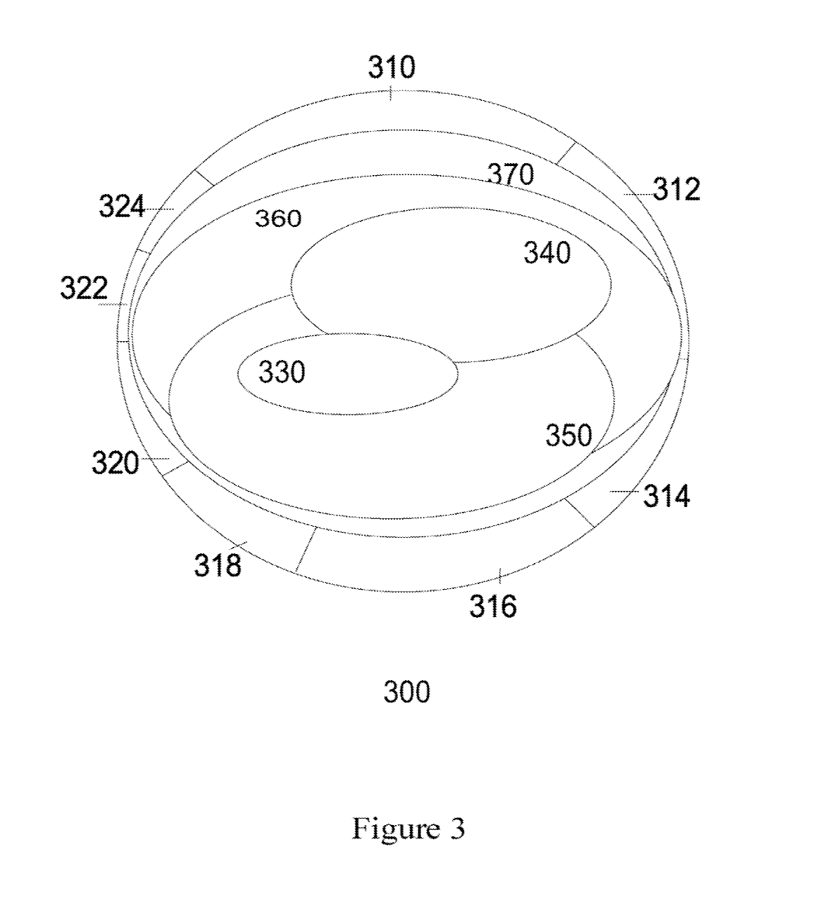

This method rests on the ability to redefine and represent various diseases, including cancer, inflammatory disorders, autoimmune diseases, neurological disorders as diseases of perturbed pathway activity. Various molecular genetic lesions or variations characteristic for specific diseases are the cause of specific pathway alterations, and these, in turn, are the mediators of the disease phenotype. As shown in FIG. 2, cancers 200 can be classified based on the specific cancer type as prostrate 210, lung 212, breast 214, colorectal 216, endometrial 218, sarcomas 220, leukemia 222, and other solid 224. Alternatively, as shown in FIG. 3, this classification of cancers (as prostrate 310, lung 312, breast 314, colorectal 316, endometrial 318, sarcomas 320, leukemia 322, and other solid 324) can be overlaid with a new classification based on the various pathway alterations that are involved in a pathway including JAK-STAT 330, Src 340, IKK-NFkB 350, Ras-Raf-ERK 360, Core PI3′K 370. Likewise, inflammatory, autoimmune, and neurological disorders can be classified based on specific pathway alterations and perturbations.

Most available information about the involvement of certain pathway alterations in specific diseases stem from laboratory and clinical molecular and genetic studies, supplemented by a growing amount of information from genetic and proteomic systematic studies and databases. Based on this information the key pathway alterations involved in major diseases have been identified, and can be modeled in engineered or naturally occurring cells and cell lines. This collection of engineered and natural cells and cell lines are generated to cover most known pathway alterations involved in specific diseases, and they are at the core of the approach.

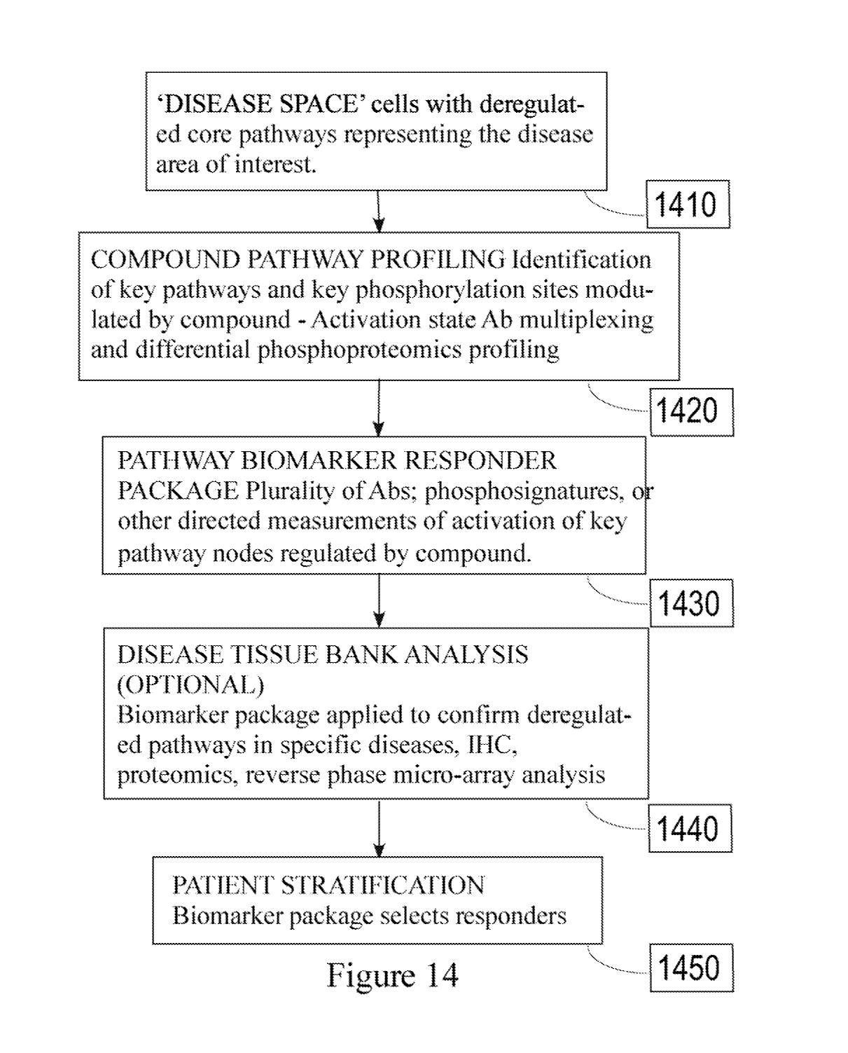

FIG. 14 shows an embodiment of the invention, where a representative flow scheme can be used to identify and generate pathway-based biomarkers for a specific therapeutic against a disease, where the disease is first identified 1410, and next compound action against disease pathways is profiled 1420, next the biomarker responder package is selected 1430, and the patient stratification involving steps 1410-1450. Step 1440 is optional where the biomarker responder package 1430 is applied in tissue to confirm the deregulated or non functional pathway in a mammal with the disease.

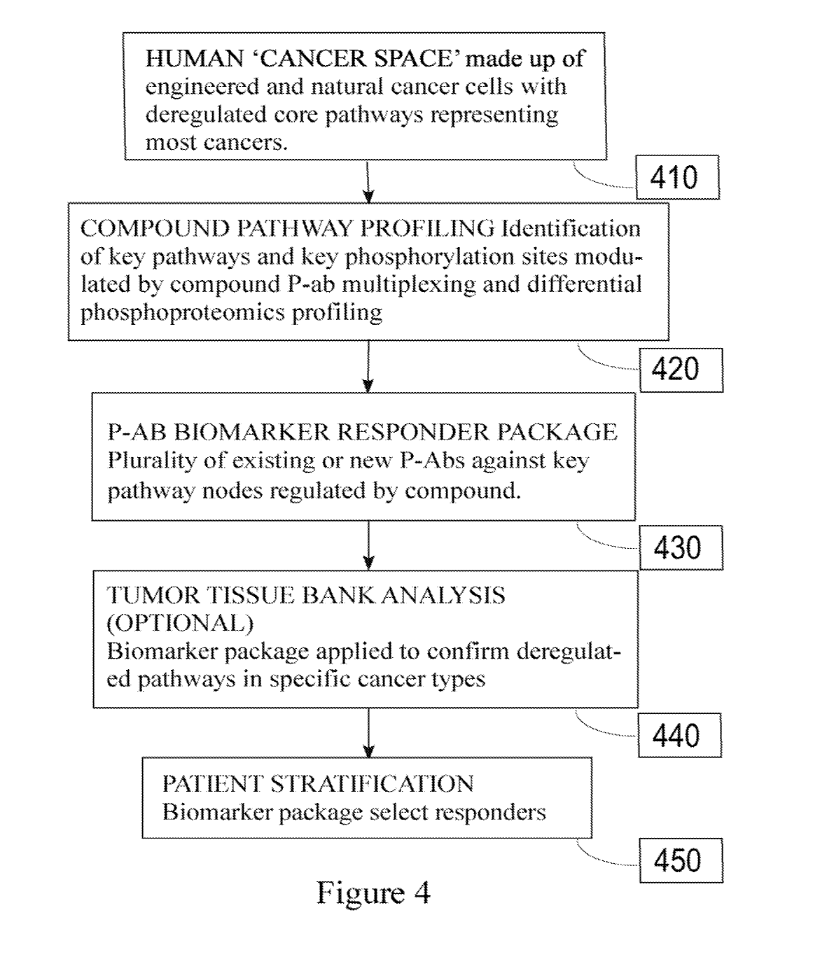

FIG. 4 shows an embodiment of the invention, where a representative flow scheme can be used to identify and generate pathway-based biomarkers for a specific therapeutic, where the cancer space is first identified 410, and next compound action against disease pathways is profiled 420, next the phosphoantibody responder package is selected 430, and the patient stratification involving steps 410-450. Step 440 is optional where the antibody responder package 430 is applied in tissue to confirm the deregulated or non functional pathway in the specific form of cancer.

### I. Disease Space Coverage

In the first step, cell lines are obtained or generated from cells that have specific core pathway alterations relevant for a certain disease. For instance, deregulated core phosphoinositide 3′ kinase (PI3′K) signaling is causally involved in a major fraction of solid and hematopoietic cancers. A number of genetic gain-of-function (GOF) and loss-of-function (LOF) mutations in human cancer cause deregulated core PI3′K signaling. These include, but are not limited to LOF of the tumor suppressor PTEN, GOF mutations of PI3′K, either through mutations in the regulatory or catalytic subunits of PB′K, amplifications and GOF mutations of the serine/threonine protein kinase PKB, also called Akt, amplifications of the serine/threonine protein kinase p70S6K, LOF of the tumor suppressor protein TSCI/2. Accordingly, human cells and cell lines are engineered to harbor these GOF and LOF mutations through (inducible) cDNA over expression (GOF mutations) or (inducible) knock down (LOF) through usage of inducible, lentiviral shRNA directed against the specific mRNA. As a control, the same cell line that these mutations are introduced into, can be kept unmodified, as a matched pair control. In addition, a number of human cancer cell lines have been identified and isolated from human cancer patients with deregulated PB′K signaling, so these naturally-occurring cancer cell lines can be part of the cellular repertoire to cover relevant PI3′K pathway alterations. By extension of this approach major cancer core pathways known to be relevant for specific cancers, including, but not limited to canonical Ras-Raf-MAPK signaling (a number of solid and hematopoietic cancers have deregulated Ras signaling), deregulated JAK-STAT signaling (numerous hematopoietic malignancies and myeloproliferative disorders), deregulated Src kinase signaling (hematopoietic malignancies), deregulated IKK-NFkB signaling (multiple myeloma, plasma cell disorders, other hematopoietic malignancies, liver carcinoma) can be addressed. In addition, relevant mutations in cell surface proteins and receptors, in particular in receptor protein tyrosine kinases, will be modeled in the cell lines. Most pathways relevant for cancer in mammals, so-called ‘cancer space’ (FIGS. 2 and 3) can be determined. By linking specific pathway alterations with specific mammalian forms of cancer, and have these pathway alterations modeled into cell lines and cells, most forms of cancer can be represented.

The same approach is used to generate cell lines and cells with deregulated pathway alterations relevant for other diseases, and hence representing the disease space for the particular type of disease under investigation, e.g. inflammatory, autoimmune, neurological. Finally, the custom-engineered and natural cells and cell lines representing the disease space are carefully characterized to ensure that they have the expected and proper pathway deregulation. This is primarily done by phosphopathway analysis using commercially available phosphor-antibodies (P-Abs). A vast number of P-Abs directed against specific phosphoproteins involved in deregulated core pathways have been generated over the years, and they cover the major signaling pathway nodes. In various embodiments of the invention, each patient population suffering from a disease where a cell line collection can be used to identify pathways of action of an exogenous agent can be determined.

### II. Compound Pathway Profiling

The cellular modeling of disease space by deregulated pathways will enable the identification and measurement of the effects of a particular therapeutic agent on specific pathways. This can be done through P-Ab multiplexing with P-Abs, phosphoproteomics analysis to identify phosphosignatures, and other probes for measuring pathway activity. This information, in turn, is useful for a number of purposes including:

a. confirmation of the suspected on-target(s) for the therapeutic by the expected pathway effects;

b. identification of potentially unknown ‘off-target’ activity by effects on pathways that are not related to the known ‘on-target(s)’. This information can be crucial in identifying potentially new therapeutic area opportunities, through the connection between specific pathways and specific diseases, based on the above pathway representation and definition of disease;

c. identification of dynamically regulated phosphosignatures or other post-translational pathway modifications. These, in turn, are the basis for generation of dynamic pathway biomarkers, e.g. phosphoantibodies, directed phosphoproteomics measurements, and other directed pathway ‘probes’;

d. identification of pathways that are not affected by the therapeutic. Through the causal association of these pathways with specific diseases, this enables the generation of so-called exclusion pathway biomarkers. These are pathway probes, e.g. phosphoantibodies or other activation-state antibodies, directed phosphoproteomics, or other measurements of the pathway activity that the therapeutic agent is inactive against. To the extent that these pathways are involved in the disease phenotype, exclusion biomarkers can be applied to exclude patients with this pathway activity from the specific treatment, since the agent is inactive against these.

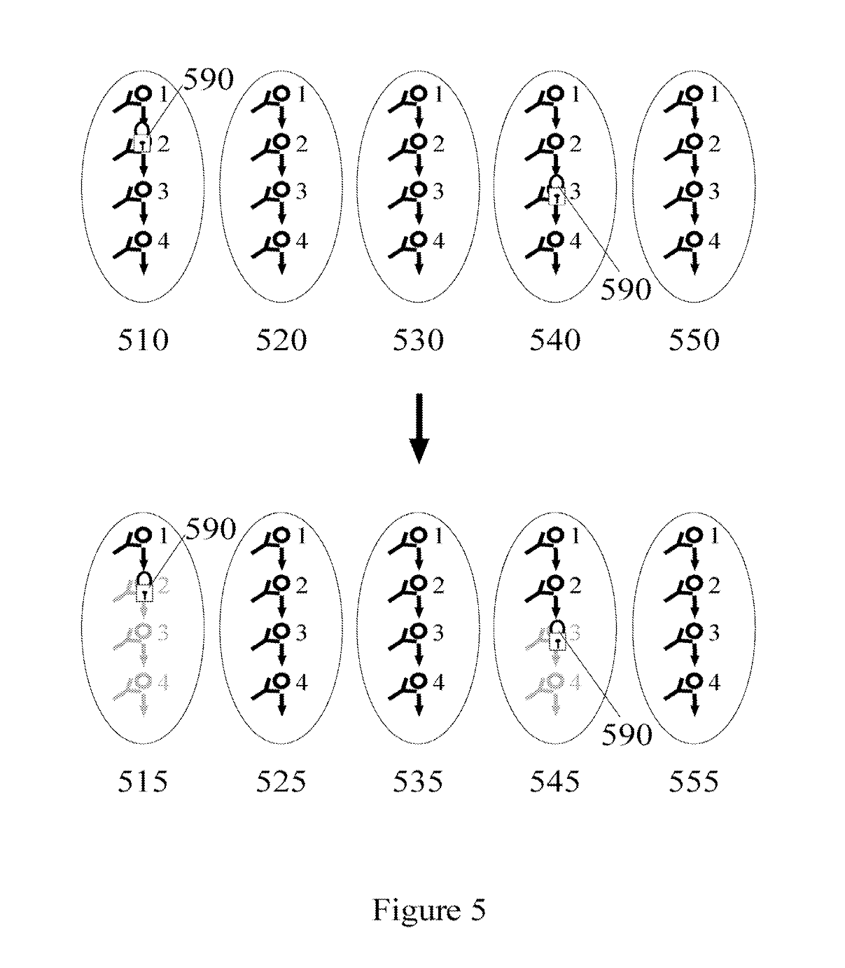

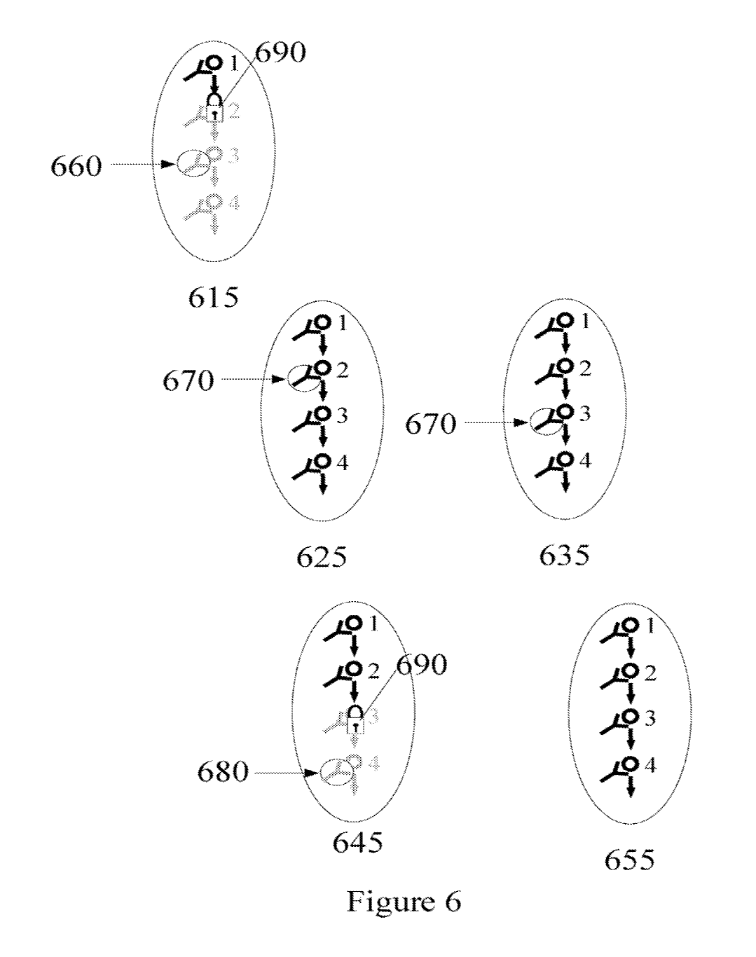

An example of compound profiling in a Disease Space, where the P-TEN-PI3′K 510, 610, 710, 810 Ras-Raf-ERK 520, 620, 720, 820 IKK-NFkB 530, 630, 730, 830 JAK-STAT 540, 640, 740, 840 and Src 550, 650, 750, 850 pathways are shown in FIGS. 5-8. A compound 590, 690, 790, 890 that is known to inhibit a PI3′K pathway 515, 615, 715, 815 target is used as an example. As illustrated in FIG. 6, the compound 690 is confirmed to hit the PI3′K pathway 615, resulting in decreased phosphorylation distal in the P13′K pathway, as measured with P-Abs 660. The compound does not effect the Ras-Raf-ERK 525, 625, 725, 825 IKK-NFkB 535, 635, 835 and Src 555, 655, 755, 855 pathways. However, the compound is shown to also interfere with core JAK-STAT pathway signaling 545, 645, 745, 845 as measured with P-Ab 680. In an embodiment of the invention, this profiling can identify a potential new disease indication for the compound, namely diseases where perturbed JAK-STAT signaling 645 is involved. The dynamic and exclusion biomarkers can be derived from dynamic P-Abs 660 and/or exclusion phosphoantibodies 670.

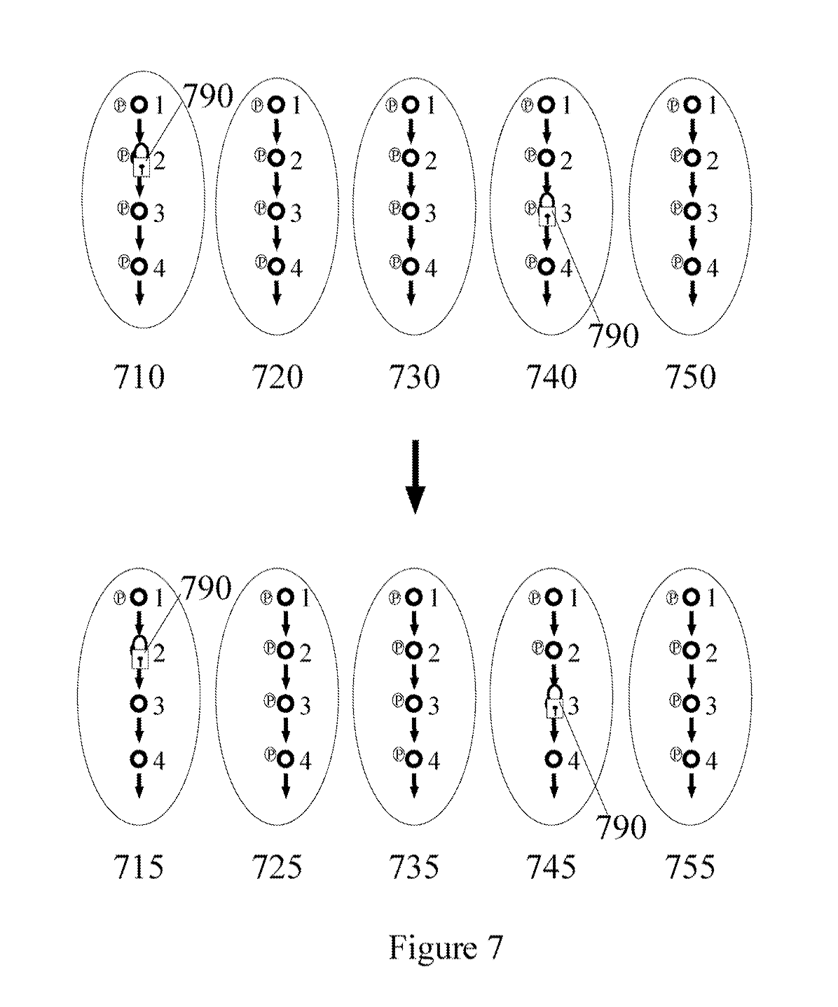

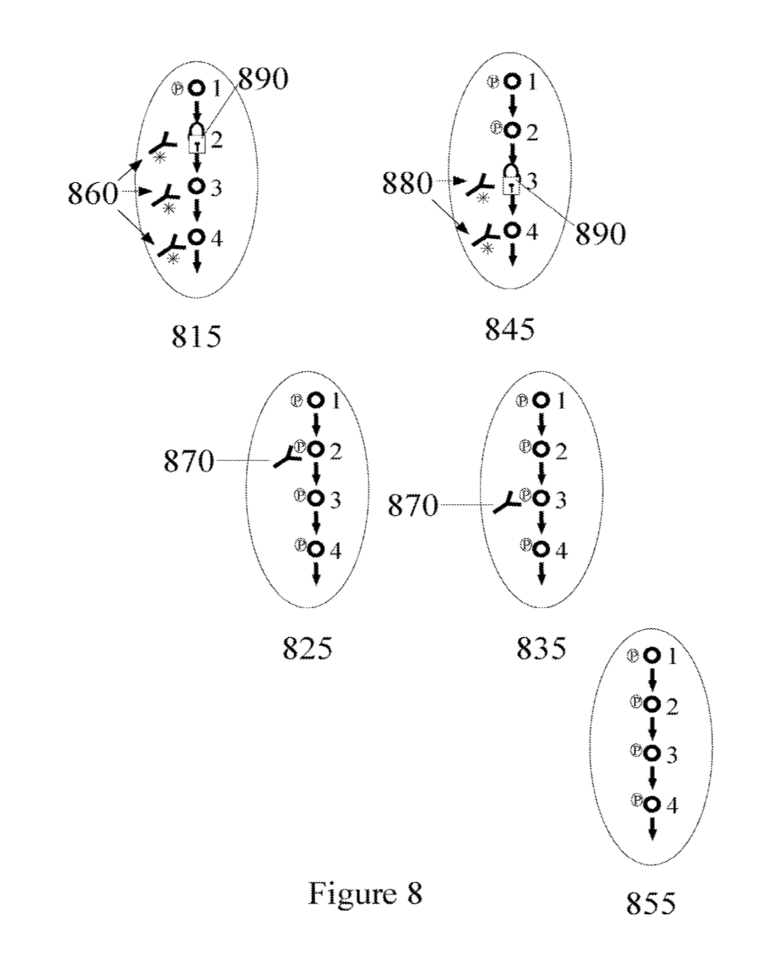

In an alternative embodiment of the invention, illustrated in FIGS. 7 and 8, the compound 790, 890 is confirmed to hit the PI3′K pathway 715, 815 resulting in decreased phosphorylation distal in the P13′K pathway, as measured with directed differential phosphoproteomics 860. However, the compound is also shown to interfere with core JAK-STAT pathway signaling 745, 845 as measured with directed differential phosphoproteomic.s 880. In an alternative embodiment of the invention, this profiling can identify a potential new disease indication for the compound, namely diseases where perturbed JAK-STAT signaling 745, 845 is involved. The dynamic and exclusion biomarkers can be derived from dynamic phosphosignatures 860, 880 and exclusion phosphosignatures 870.

### III. Pathway Biomarker Responder Package

The panel of dynamic and exclusion biomarkers together constitutes a ‘biomarker package’ that when used together on diseased tissue will enable a rational prediction of therapeutic efficacy by the specific therapeutic agent that was profiled (FIGS. 6 and 8). In essence, this is a simple, custom-generated predictive and response biomarker package, consisting of a panel of biomarkers tailored for the therapeutic agent. The package will be validated by application to the cellular model of disease space to confirm the expected pathway alterations. Once validated, this biomarker package can be applied on disease tissues and on biopsies from patients or sick animals entering clinical trials to ensure segregation of responder s from non-responders, as described in IV and V below.

### IV. Disease Tissue Bank Analysis

The pathway biomarker responder package can be applied to relevant human or animal disease tissue banks. Typically these are paraffin embedded, more rarely cryo-preserved after OCT mounting. The purpose of this is to confirm that the perturbed pathway activity pattern that is specifically measured with the biomarker package is recognized in the relevant disease tissue. In particular one or two of the biomarkers in the biomarker package, which can consist of 4-8 predictive biomarkers, might not confirm that the particular pathway activity is perturbed as in the cellular model of the disease space. This information can be used to go back and repeat steps I to III above to identify additional biomarkers to replace the non-confirmatory biomarkers. While there can be many reasons for such a lack of confirmation, the most likely is that the cells are grown artifactually in two dimensions on a plastic dish, and hence many signaling pathways are not regulated as in adherent cells growing as part of the disease tissue. This caveat is difficult to overcome, but one way to partially overcome this is through analysis of a number of cell lines and cells where the same pathway perturbation is achieved through different genetic alterations relevant for the disease of interest.

### V. Patient Stratification

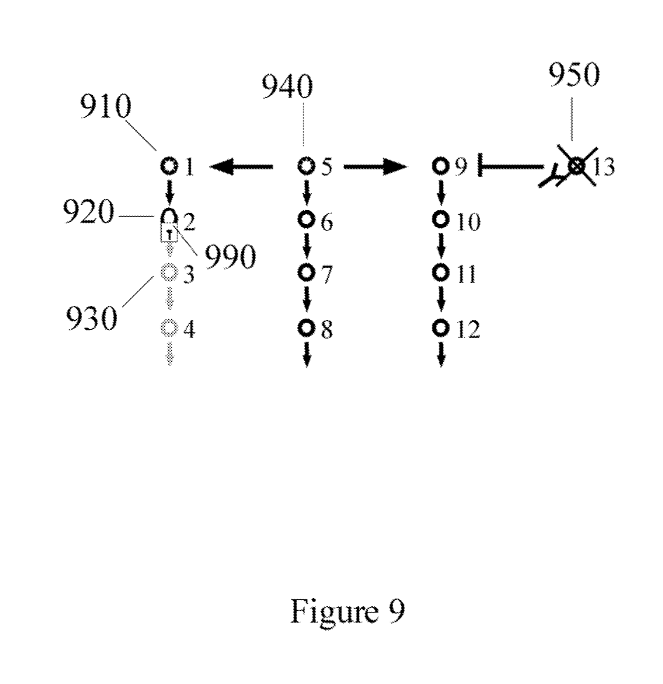

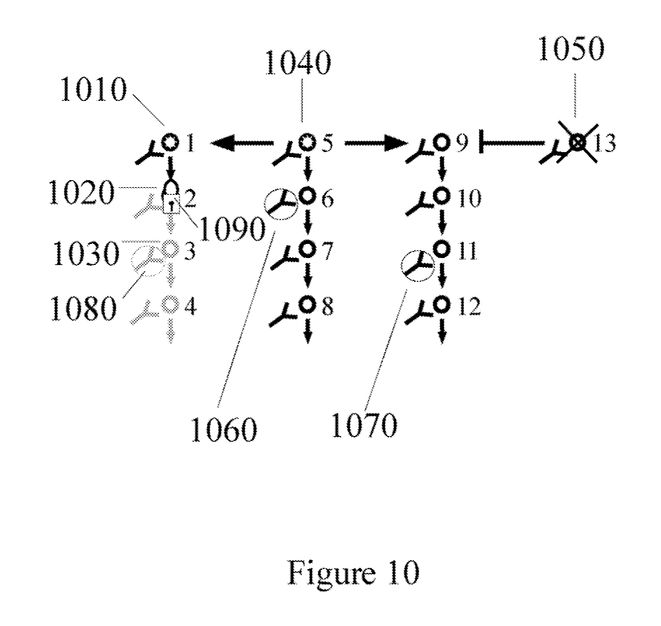

The ultimate goal of the generated biomarker responder package is to be able to apply it to patient tissue to select the patients that will respond to the specific therapeutic agent. The biomarker package is purposely as simple as possible with the highest predictive power such that it can be used to stratify patients in early clinical trials and also be marketed hand-in-hand with the specific therapeutic agent. In clinical trials, the biomarker package will ideally be applied on diseased tissue before and after treatment with the therapeutic agent it was developed for, so that Bayesian principles can be applied to further improve its predictive power even from a very small, yet stratified patient material. As an example, see FIGS. 9 and 10. Malignant melanoma is a cancer originating in pigment cells, so-called melanocytes, of the skin. Over 66% of malignant melanoma patients have been found to harbor a GOF B-Raf (V600E) mutation 910, 1010 rendering the serine/threonine kinase B-Raf constitutively active. This particular mutation will result in deregulated signaling through MEK 920, 1020 and ERK 930, 1030 and so in principle patients with GOF mutation of B-Raf should be responsive to a MEK inhibitor 990, 1090, and the dynamic biomarker applied to monitor MEK inhibitor target exposure is a phoshoantibody directed against the substrate of MEK, called ERK 1080. Accordingly, in clinical trials where malignant melanoma patients have a GOF-B-Raf mutation as the sole mutation, a number of patients exhibit disease stabilization and even partial regression upon MEK inhibitor treatment. However, a number of patients with GOF B-Raf mutations have concurrent GOF Ras mutations 940 and/or concurrent LOF PTEN mutations 950. These mutations result in deregulated PB′K signaling, as measured with the P-Ab against target 11, and deregulated GTPase signaling, as measured with the P-Ab against target 6 1060. Since these pathways are themselves involved in malignant transformation of cells and cancer, it is important to exclude patients with these pathways active, since the MEK inhibitor does not act on these. Accordingly, an example of how a derived P-Ab biomarker package can be used to identify responder patients for a specific MEK inhibitor for malignant melanoma, is illustrated in Table I. In an embodiment of the invention, in pre-treatment biopsies patients with high signals from exclusion P-Abs against targets 6 and 11 1060 and 1070 are excluded from treatment, while patients with high signals from P-Ab against P-ERK 1080 are included for treatment. In an alternative embodiment of the invention, in addition to the predictive and dynamic biomarkers, one or more additional biomarker can be used involving target-directed PCR against the main known mutated target genes, namely b-raf, ras, and p-ten to confirm that the biomarker package is applied to a disease with the relevant key mutations involved in the perturbed pathway alterations.

TABLE I
Stratification Principle based on antibody assay for determining
Responder Patient Population

| Antibody # | | | | |
|---|---|---|---|---|
| (from FIG. | Label in | Pre Treatment | Post Treatment | Exclude |
| 10) | FIG. 10 | Assay | Assay | Patients |
| 3 | 1080 | +++ | (+) | none |
| 6 | 1060 | (+) | (+) | ++ or +++ |
| 11 | 1070 | (+) | (+) | −+ or +++ |

### Method II

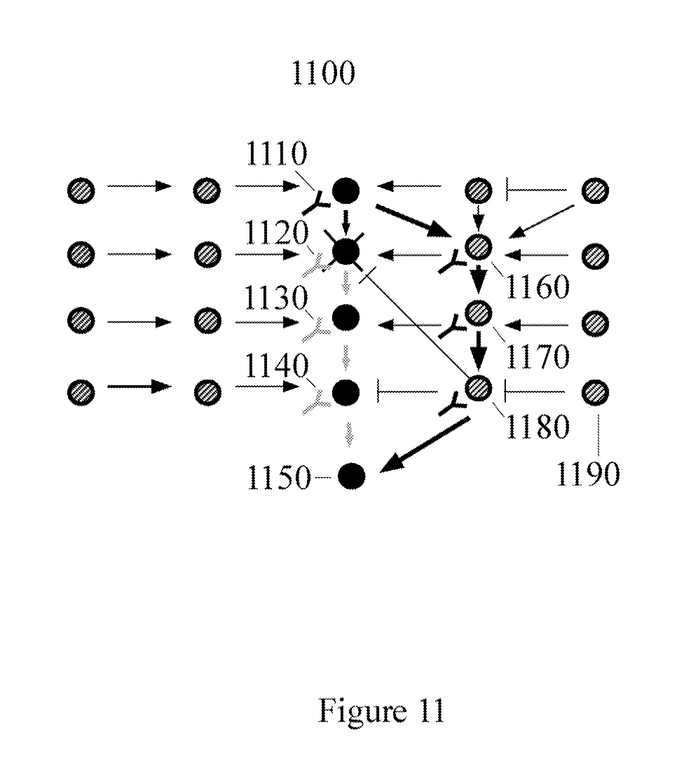

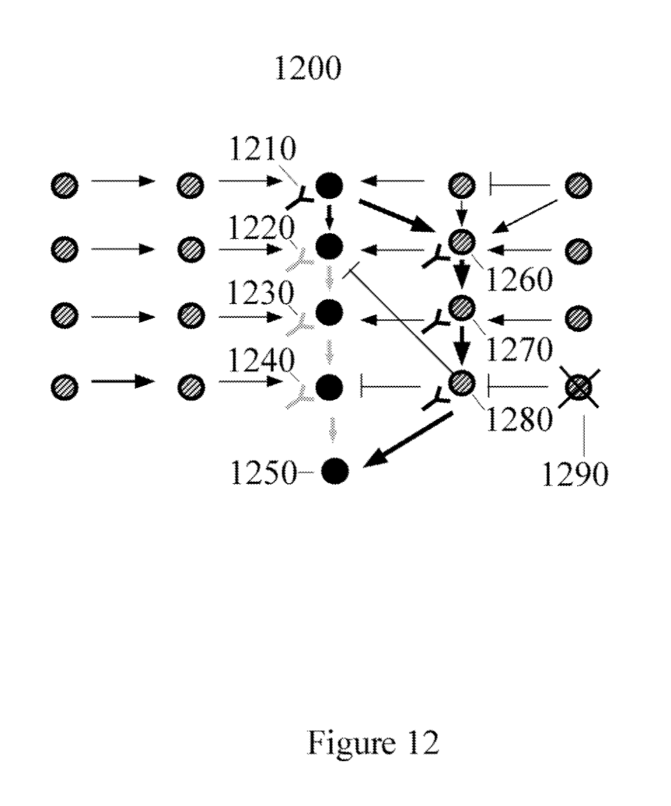

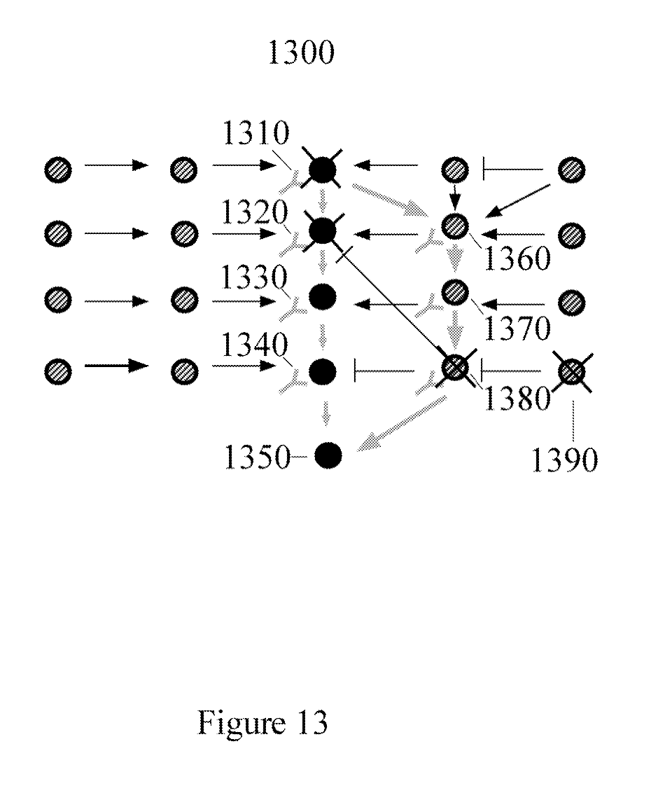

Identification of optimal target combinations with associated predictive biomarkers for diseases. The cancer space coverage by the collection of pathway context cell lines and cells can also be used to identify optimal target combinations to inhibit or modulate simultaneously to prevent by-pass pathway activity and to derive the optimal biomarker package for agents hitting such target combination. The starting point is based on defining a particular pathway of interest based on its involvement in and relevance for a particular disease(s). For instance, if the disease of interest is cancer, optimal target combinations for the various mutations that result in deregulated core PB′K and Ras-Raf-MAPK signaling could be the focus. A number of the cell lines in the disease space collection will harbor these deregulated pathways 1100, 1200, 1300, see FIGS. 11-13. Cell lines containing a particular pathway perturbation of interest are interrogated because of their relevance for a disease of interest. Through multiparameter P-Ab analysis, differential global phosphoprofiling, or other pathway read-outs, the overall pathway activity inside the cells of interest are monitored. To assess whether a particular target, e.g. target 2 in this example, is a potential attractive target for inhibition of deregulated pathway activity, that particular target is inactivated through (inducible) knock down. The LOF in essence mimicks a therapeutic agent targeting that protein. As shown in FIG. 11, as a consequence of the target inhibition of inhibited pathway 1110-1150 a ‘rescue’ or ‘by-pass’ pathway 1160-1180 is immediately activated, as measured with the pathway monitoring approach. Similarly, as shown in FIG. 12, inhibition of a target not involved in the pathway 1190 does not alter the status quo and either the initial pathway 1210-1250 or a rescue pathway 1260-1280 can be activated. This pathway activation is undesirable if it can potentially be involved in a disease phenotype. In an embodiment of the invention, in order to prevent this by-pass mechanism from kicking in, systematic testing of each of the core pathway targets through (inducible) knock down individually, followed by multiparameter pathway analysis can be carried out. An optimal combination of the directed target knock downs can be achieved (see FIG. 13) when they result in quenching of major pathway activity 1310-1380 irrespective of whether they also knock down other targets 1390. Based on the same principle as used above to derive biomarkers for specific therapeutic agents, the optimal biomarker package to be used for therapeutic combinations that would hit the optimal combination of targets can be derived. Based on the identification of the optimal target combinations through mimicking of a therapeutic through genetic knock down, this method is particularly attractive for RNAi therapeutics. Assuming that the delivery issue for RNA-based therapeutics will soon be solved, this would be an ideal method for identifying optimal target combinations for RNA-based therapeutic cocktails for specific diseases, and generate the optimal associated biomarker package for these.

### Method III

Pathway interceptor screening approach for identification of clinically relevant new targets for specific diseases. In another embodiment of the present invention, new targets relevant for a specific disease can be identified using bioinforrnatics-derived target libraries optimized for the likelihood of targets that interact with the core deregulated pathway causing a disease of interest. Through inducible, lentiviral shRNA knock down of all targets in the library the effects of this down regulation is measured through phosphopathway analysis by phosphoantibody multiplexing of the core deregulated pathways. Targets that when inducibly knocked down cause decreased signaling through the core deregulated disease pathway modeled in the cell line(s) of study, will be candidates for clinically relevant therapeutics development for said disease. As the screening approach is outlined in PCT Application WO/2005/103299 “RNAi-Based Target Identification and Validation”, inventor: Blume-Jensen, P.) which is expressly incorporated by reference in its entirety.

In an embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In an embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In an alternative embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, wherein one or more of the signaling pathways is active, assaying the state of the one or more signaling pathway with the biomarker responder package before and after drug dosing and identifying the responder patient population that will be responsive for the exogenous agent based on the assay.

In a another embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the biomarker responder package contains between: a lower limit of three and an upper limit of twenty biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In a further embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the biomarker responder package contains one or more exclusion biomarkers and one or more dynamic biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In an embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the biomarker responder package contains one or more predictive biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In another embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay.

In an alternative embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay, wherein the assay detects one or more signaling pathways that are down regulated by drug treatment, wherein based on the assay, patients are excluded from the responder patient population based on the inability of the drug to modulate said disease pathways.

In an embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay, wherein the assay detects one or more disease-relevant signaling pathways that are up regulated.

In various embodiments of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, assaying the state of the one or more signaling pathway with the biomarker responder package before and after drug dosing and identifying the responder patient population that will be responsive for the exogenous agent based on the assay, wherein the assay detects one or more disease-relevant signaling pathways that are up regulated, wherein based on the assay patients are included in the responder patient population.

In an embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, wherein the biomarkers are pathway activity state biomarkers, assaying the state of the one or more signaling pathway with the biomarker responder package before and after drug dosing and identifying the responder patient population that will be responsive for the exogenous agent based on the assay.

In another embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, wherein the biomarkers are antibodies directed against post-translationally modified proteins, assaying the state of the one or more signaling pathway with the biomarker responder package before and after drug dosing and identifying the responder patient population that will be responsive for the exogenous agent based on the assay.

In a further embodiment of the invention, a method of identifying a responder patient population for treatment with an exogenous agent comprises: establishing a cellular model of disease space based on one of more signaling pathway, identifying the effect of the exogenous agent in the one or more signaling pathway, determining a biomarker responder package including a plurality of biomarkers, wherein the plurality of biomarkers are specific for one or more of the signaling pathway, wherein the biomarkers are antibodies directed against phospho proteins, assaying the state of the one or more signaling pathway with the biomarker responder package before and after drug dosing and identifying the responder patient population that will be responsive for the exogenous agent based on the assay.

Although the present invention has been shown and described in detail with regard to only a few exemplary embodiments of the invention, it should be understood by those skilled in the art that it is not intended to limit the invention to the specific embodiments disclosed. Various modifications, omissions, and additions may be made to the disclosed embodiments without materially departing from the novel teachings and advantages of the invention, particularly in light of the foregoing teachings. Accordingly, it is intended to cover all such modifications, omissions, additions, and equivalents as may be included within the spirit and scope of the invention as defined by the following claims.

What is claimed is:

1. A method of identifying a cancer type of interest, comprising:
(a) establishing a cellular model of cancer space based on two or more cell lines each having one or more disease signaling pathways causally involved in cancer type of interest;
(b) treating the two or more cell lines with one or more exogenous agents;
(c) using phosphor-antibodies (P-Abs) to analyze phosphopathways of one or more biomarkers before and after treatment with the exogenous agents;
(d) determining the effect of the exogenous agents on an activity state of the one or more disease signaling pathways in the cell lines by identifying at least one of: (i) abundance of one or more biomarkers, and (ii) post-translational modifications of one or more biomarkers, before and after treatment with the one or more exogenous agents through phosphopathway analysis using phosphor-antibodies (P-Abs);
(e) deriving one or more biomarkers for an activity state of the one or more disease signaling pathways in the cell lines from the one or more biomarkers; and
(f) defining the cancer type based on the one or more disease signaling pathways and responsiveness of the pathways to the one or more exogenous agents.

2. The method of claim 1, wherein the cancer type of interest is a new or previously unknown cancer type with respect to the disease pathway.

3. The method of claim 1, wherein the post-translational modification is phosphorylation.

4. The method of claim 1, wherein the post-translational modification is cellular modification of a protein.

5. The method of claim 1, wherein the phosphopathway analysis is selected from the group consisting of differential phosphoproteomics profiling to identify phosphorsignatures, phosphoantibody multiplexing, reporter assays, degradation of signaling proteins by ubiquitination, other methods of post-translational proteomics profiling by mass spectrometry, and other proteasome-mediated processes.

6. The method of claim 1, wherein at least one of the one or more disease signaling pathways is selected from the group consisting of P-TEN-PI3′K 510, 610, 710, 810 Ras-Raf-ERK 520, 620, 720, 820 IKK-NFkB 530, 630, 730, 830 JAK-STAT 540, 640, 740, 840 and Src 550, 650, 750, 850 pathways.

7. A method of classifying cancer, comprising:
identifying an alteration involved in a disease pathway.

8. The method of claim 7, wherein the alteration is selected from the group consisting of JAK-STAT 330, Src 340, IKK-NFkB 350, Ras-Raf-ERK 360, and Core PI3′K 370.
